(19) United States
(12) Patent Application Publication
(10) Pub. No.: US 2020/0208194 A1
(43) Pub. Date: Jul. 2, 2020

(54) COAGULATION AND FIBRINOLYSIS ASSAYS

(71) Applicants: Massachusetts Institute of Technology, Cambridge, MA (US); University of Colorado, Denver, CO (US)

(72) Inventors: Michael B. Yaffe, Cambridge, MA (US); Chris Barrett, Walpole, MA (US); Ernest Moore, Denver, CO (US); Hunter Moore, Denver, CO (US)

(73) Assignees: Massachusetts Institute of Technology, Cambridge, MA (US); University of Colorado, Denver, CO (US)

(21) Appl. No.: 16/612,569

(22) PCT Filed: May 11, 2018
(86) PCT No.: PCT/US2018/032335

§ 371 Date: Nov. 11, 2019

Related U.S. Application Data
(60) Provisional application No. 62/505,021, filed on May 11, 2017.

(51) Int. Cl. C12Q 1/56; G01N 33/49; G01N 33/86

(52) U.S. Cl. CPC C12Q 1/56; G01N 33/4905; C12Q 2565/629; G01N 2333/8121; G01N 2333/968; G01N 33/86

(57) ABSTRACT

Coagulation and fibrinolysis assays and related compositions, systems, methods, and kits are provided. In some embodiments, a coagulation and fibrinolysis assay may utilize one or more biological molecules. For instance, the assay may comprise combining a blood or blood-derived patient sample with the biological molecule(s) and measuring one or more properties of the sample associated with coagulation and/or fibrinolysis. The biological molecules may serve to shorten the assay duration and/or enhance the sensitivity of the assay relative to certain conventional assays. In certain embodiments, the biological molecules may allow pathological coagulation and/or fibrinolysis phenotypes to be elucidated. The coagulation and fibrinolysis assays described herein may be used for a wide variety of clinical and/or laboratory applications, including the diagnosis of certain coagulation and/or fibrinolysis disorders, such as trauma-induced coagulopathy and hyperfibrinolysis.

### RELATED APPLICATIONS

This application claims priority under 35 U.S.C. § 119(e) to U.S. Provisional Patent Application Ser. No. 62/505,021, filed May 11, 2017, and entitled “Modified Coagulation Assay that Rapidly Unmasks Pathological Fibrinolysis Phenotypes in a Wide Spectrum of Human Diseases,” which is incorporated herein by reference in its entirety for all purposes.

### TECHNICAL FIELD

Coagulation and fibrinolysis assays and related compositions, systems, methods, and kits associated therewith are generally described.

### BACKGROUND

Traumatic injury is the leading cause of death between ages 1-44, resulting in 180,000 deaths annually in the U.S. Early preventable death (<12 hours) after injury generally results from excessive bleeding/hypocoagulability, while late death (5-14 days) results from infection and organ failure, where microvascular thrombosis and/or hypercoagulability plays a major role. Unfortunately, current clinical and/or laboratory tests provide incomplete, static information on blood clotting based largely on in vitro solution-phase assays of clotting proteins in the absence of cells. In addition, these tests provide little to no information on the entire latter half of the coagulation system (fibrinolysis) that often drives persistent bleeding or thrombosis after trauma, surgery, and other pathological states. This generally makes prompt therapeutic interventions aimed at correcting abnormal fibrinolytic activity a “best-guess,” which is problematic in multiple clinical settings including, but not limited to trauma, where patients frequently have multiple clotting abnormalities (including deranged fibrinolysis) and the wrong treatment at the wrong time is usually harmful and potentially lethal. Thus, a critical gap in current medical technology is the ability to rapidly, accurately and functionally assess blood clotting in a way that includes rapid and useful information on fibrinolysis.

Under normal conditions, blood clotting results in rapid formation of a hemostatic clot of limited size. Proper control of coagulation is accomplished by localized clot activation and by clot breakdown (i.e., fibrinolysis) that protects organs and body parts from ischemia by “policing” the vasculature for unintended or harmful clotting. Trauma, sepsis, and other pathological states such as liver disease and solid organ transplantation often result in too much fibrinolysis (termed “hyperfibrinolysis”) relative to the clots that are being formed, leading to bleeding complications and hemorrhagic death. On the opposite end of the spectrum, many injured and/or critically ill patients develop an inability to appropriately breakdown clots that may form in their vasculature (termed “fibrinolysis shutdown”), which can lead to organ failure and death. In trauma alone it is estimated that up to 80% of all preventable deaths early after major injury result directly from bleeding. Current measurements of blood clotting in widespread clinical use (the Protime and Partial Thromboplastin time), however, are poorly reflective of clinical bleeding and thrombosis because they examine clot formation in a static artificial non-functional environment while ignoring both the second half of the coagulation system (i.e., fibrinolysis) and the functional contribution of blood cells such as platelets. There is a growing trend in hospitals to use functional viscoelastic assays, such as thromboelastography (TEG) and rotational thromboelastometry (ROTEM), in order to examine the entire process of clot formation and fibrinolysis in whole blood and plasma products. Other microfluidic devices are now being introduced as an additional method to study clot formation and fibrinolysis in a functional manner. However, a major limitation of all these current technologies is that they typically require 60-90 minutes before a result on fibrinolysis activity is obtained, and the magnitude of the differences between normal and hyperfibrinolytic patients, as well as fibrinolysis shutdown patients using the current assays are often very small, but clinically very significant. This relatively long time delay in obtaining marginally useful test results required for diagnosis is particularly problematic in a massively bleeding hyperfibrinolytic patient, for example, where rapid clinical decision making regarding anti-fibrinolytic therapies and blood product administration is virtually impossible to accomplish in a timely manner using currently available assays. Accordingly, improved compositions and methods are needed.

### SUMMARY

Coagulation and/or fibrinolysis assays and related compositions, systems, methods, and kits associated therewith are provided.

The subject matter of the present invention involves, in some cases, interrelated products, alternative solutions to a particular problem, and/or a plurality of different uses of one or more systems and/or articles.

In one set of embodiments, coagulation and fibrinolysis assay compositions are provided. In one embodiment, a coagulation and fibrinolysis assay comprises a sample being tested and plasmin and/or plasminogen that is exogenous with respect to the sample being tested.

In another embodiment, a coagulation and fibrinolysis assay composition comprises a sample being tested and an exogenous sequestering agent.

In another set of embodiments, coagulation and fibrinolysis assay kits are provided. In one embodiment, a coagulation and fibrinolysis assay kit comprises a sample collection container free of a subject sample for testing and configured for containing the subject sample and an exogenous plasmin and/or plasminogen.

In another embodiment, a coagulation and fibrinolysis assay kit comprises sample collection container free of a subject sample for testing and configured for containing the subject sample, an exogenous sequestering agent, and a clotting activator.

In one set of embodiments, methods are provided. In one embodiment, a method comprises combining a blood or blood-derived sample from a patient being tested with a plasmin and/or plasminogen that is exogenous with respect to the blood or blood-derived sample and performing a coagulation and fibrinolysis assay on the sample.

In another embodiments, a method comprises combining a blood or blood-derived sample from a patient being tested with a sequestering agent that is exogenous with respect to the blood or blood-derived sample and performing a coagulation and fibrinolysis assay on the sample.

In yet another embodiment, a method comprises combining a blood or blood-derived sample from a patient being tested with a plasmin and/or plasminogen and a tissue plasminogen activator that are exogenous with respect to the blood or blood-derived sample and performing a coagulation and fibrinolysis assay on the sample.

In one aspect, the present disclosure describes a new test that measures fibrinolysis/clot lysis. In certain embodiments, the test can be performed and results returned much more rapidly than other conventionally employed known methods in clinical blood samples (including whole blood, anticoagulated whole blood, plasma, and anticoagulated plasma). In certain embodiments, a new reagent-based diagnostic test is provided that allows for performance of embodiments of the disclosed test in functional coagulation assay machines including those performing viscoelastic assays and microfluidics assays.

In certain embodiments, a new reagent-based diagnostic test is provided that can overcome some or all of the above described limitations of typical conventional tests and that can rapidly and robustly identify pathological hyperfibrinolysis for expedient therapeutic intervention to provide personalized patient care to ill, critically ill and injured patients. In certain embodiments, a new reagent-based diagnostic test is provided that can identify a cause of lower than normal amounts of fibrinolysis (“fibrinolysis shutdown”) for consideration of therapeutic intervention in ill, critically ill and injured patients. In certain embodiments, the approach is based on development, in the context of certain embodiments disclosed herein, of assays in which addition of the enzyme plasmin, and/or plasminogen, which is the precursor of plasmin, to functional coagulation assays, including viscoelastic assays and also applicable to microfluidic assays, can rapidly unmask an underlying pathological hyperfibrinolytic state (the addition of plasmin with or without addition of plasminogen) while in others can unmask a cause for fibrinolysis shutdown (the addition of plasminogen with or without plasmin).

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows a schematic of fibrinolysis;

FIG. 2 show a schematic of the fibrinolytic pathway;

FIG. 3 show a schematic of a modified fibrinolytic pathway, according to certain embodiments;

FIG. 4A shows a schematic representation of an assay, according to certain embodiments;

FIG. 4B shows a schematic representation of an assay, according to certain embodiments;

FIG. 4C shows a schematic representation of an assay, according to certain embodiments;

FIG. 5 shows a thromboelastography (TEG) trace, according to one set of embodiments;

FIG. 6 shows a rotational thromboelastometry trace, according to one set of embodiments;

FIG. 7 shows a schematic representation of a coagulation and fibrinolysis assay, according to certain embodiments;

FIG. 8 shows TEG traces with and without the addition of exogenous plasmin, according to one set of embodiments;

FIG. 9 shows TEG traces with and without the addition of exogenous plasmin before and after liver transplant, according to one set of embodiments;

FIG. 10 shows graphs of R-time versus time, angle versus time, maximum amplitude versus time, time to maximum amplitude versus time, and clot lysis at 30 minutes after maximum amplitude for various concentrations of plasmin used in a TEG assay, with the TEG traces for each concentration of plasmin also being shown;

FIG. 11 shows graphs of R-time versus time, angle versus time, maximum amplitude versus time, time to maximum amplitude versus time, and clot lysis at 30 minutes after maximum amplitude for various concentrations of plasmin used in a TEG assay, with the TEG traces for each concentration of plasmin also being shown; and

FIG. 12 shows a graph of time to maximum amplitude for various TEG assays with and without plasmin, and also shows TEG traces for assays including tissue plasminogen activator and plasmin and a table of the results.

### DETAILED DESCRIPTION

Coagulation and fibrinolysis assays and related compositions, systems, methods, and kits are disclosed. In some embodiments, a coagulation and fibrinolysis assay utilizes one or more biological molecules (e.g., plasmin, plasminogen, and inhibitors of serine protease). For instance, the assay in certain embodiments comprises combining a sample, for example a blood or blood-derived (e.g. plasma, serum, etc.) patient sample, with the biological molecules and measuring one or more properties of the sample associated with coagulation and/or fibrinolysis. The biological molecule(s) may serve to shorten the assay duration and/or enhance the sensitivity of the assay relative to certain conventional assays. In certain embodiments, the biological molecules allow pathological coagulation and/or fibrinolysis phenotypes to be elucidated. The coagulation and fibrinolysis assays described herein may, in various embodiments, be used for a wide variety of clinical and/or laboratory applications, including the diagnosis of certain coagulation and/or fibrinolysis disorders, such as trauma-induced coagulopathy and hyperfibrinolysis.

Hemostasis, the process of arresting blood from flowing out of injured vessels, is vital to human health and relies on an intricate balance between coagulation and fibrinolysis. Unfortunately, many conditions (e.g., liver disease, sepsis, cancer, traumatic injury, hemophilia) may adversely affect this balance resulting in abnormal coagulation and/or fibrinolysis. Abnormal coagulation and/or fibrinolysis may result in hemorrhage and/or thrombosis associated disorders, such as myocardial infarction, stroke, pulmonary embolism, and deep vein thrombosis, amongst others. In some people, the abnormal coagulation and/or fibrinolysis may remain latent or otherwise undiagnosed until a triggering event. In some instances, the triggering event is treatment of the condition, e.g., via surgery, blood transfusion, and/or administration of a pharmaceutically active agent. Accordingly, assessment of the hemostatic system, and accordingly coagulation and fibrinolysis, is often recommended prior to certain medical treatments, during management of acute events, and/or after the administered medical treatment(s).

Though hemostasis is a complex process that utilizes cellular, plasma, and insoluble components, many traditional hemostasis assays, such as prothrombin time, only assess certain components (e.g., plasma components) and/or portions (e.g., coagulation) of the hemostasis process. Accordingly, these traditional assays may fail to detect certain coagulation and/or fibrinolysis abnormalities, such as hyperfibrinolysis. Functional assays, such as viscoelastic assays, that aim to mimic and reflect the major physiological aspects of hemostasis process in vitro have arisen as an alternative to certain traditional assays.

However, a major limitation of conventional functional assays is the assay duration. For instance, conventional viscoelastic assays typically require 60-90 minutes before a result indicative of fibrinolysis activity is obtained. The relatively long assay duration of conventional viscoelastic assays limits the utility of functional assays in emergency situations in which clinical decisions must be made quickly. Another limitation of certain conventional functional assays is the small magnitude in the difference of assay output between normal and certain pathological conditions (e.g., hyperfibrinolysis), making differentiation between normal and abnormal results difficult. Accordingly, improved coagulation and fibrinolysis assays are needed.

The present disclosure relates to the surprising discovery that the duration and diagnostic value of functional assays may be significantly improved by utilization of biological molecules (e.g., plasmin, plasminogen, and serpin inhibitors) that are capable of sequestering (e.g., in native form, after activation) inhibitors of plasmin (collectively referred to as sequestering agents). As described in more detail below, the combination of a sample with one or more sequestering agent prior to and/or during a process (e.g., coagulation, fibrinolysis, measurement) in a functional assay may shorten the assay duration and/or improve the sensitivity. Preferred embodiments of the coagulation and fibrinolysis assays, described herein, do not suffer from one or more limitations of traditional and conventional functional hemostasis assays.

In some embodiments, a coagulation and fibrinolysis assay method may comprise combining a sequestering agent with a sample (e.g., whole blood, blood derivative). As used herein, the term “sequestering agent” refers to a biological molecule (e.g., protein, antibody, nucleic acid, aptamer, small molecule) that is capable, with or without activation, of preventing the inhibitory function of a direct or indirect plasmin inhibitor, e.g., by specifically binding the inhibitor and/or occupying or otherwise shielding (e.g., reversibly, irreversibly) the inhibitory portion of the inhibitor from interacting with a binding partner (e.g., plasmin). For example, exogenous plasmin, though having enzymatic activity, may be a sequestering agent due to the ability of the plasmin to specifically bind plasmin inhibitors. An another example, plasminogen may be a sequestering agent due to the ability to bind plasmin inhibitors upon activation by a plasminogen activator to form plasmin. As yet another example, antibodies, aptamers, nucleic acids, and/or small molecules that prevent the inhibitory function of direct plasmin inhibitors (e.g., α₂-antiplasmin and α₂-macroglobin) and indirect plasmin inhibitors (e.g., plasminogen activator inhibitors) may be sequestering agents. In some embodiments, the sequestering agent is not a plasminogen activator (e.g., tissue plasminogen activator, urokinase).

It should be understood that the predominant function of a sequestering agent in an assay may not be sequestration. In some instances, the sequestering agent in an assay, though capable, may not specifically bind a plasmin inhibitor and/or occupy or otherwise shield the inhibitory portion of the plasmin inhibitor from interacting with a binding partner. In one example, the predominant or exclusive function of exogenous plasmin at least during a portion of the assay is enzymatic cleavage of fibrin. In another example, the predominant or exclusive function of exogenous plasmin at least during a portion of the assay is cleavage and activation and/or inactivation of certain coagulation factors (e.g. Factor V). Conversely, in another example, exogenous plasmin in an assay functions to both sequester plasmin inhibitors and cleave fibrin. In general, the function of the sequestering agent in an assay depends, at least in part, on certain characteristics (e.g., concentration of inhibitors, concentration of activators, concentration of plasmin) of the sample.

The term “biological molecule” has its ordinary meaning in the art and may refer to molecule comprising sugar, amino acid, cofactor, and/or nucleotide. The biological molecule may be capable of undergoing a biological binding event (e.g., between complementary pairs of biological molecules) with another biological molecule. In some embodiments, the biological molecule may be a nucleic acid, cofactor, protein, peptide, or carbohydrate.

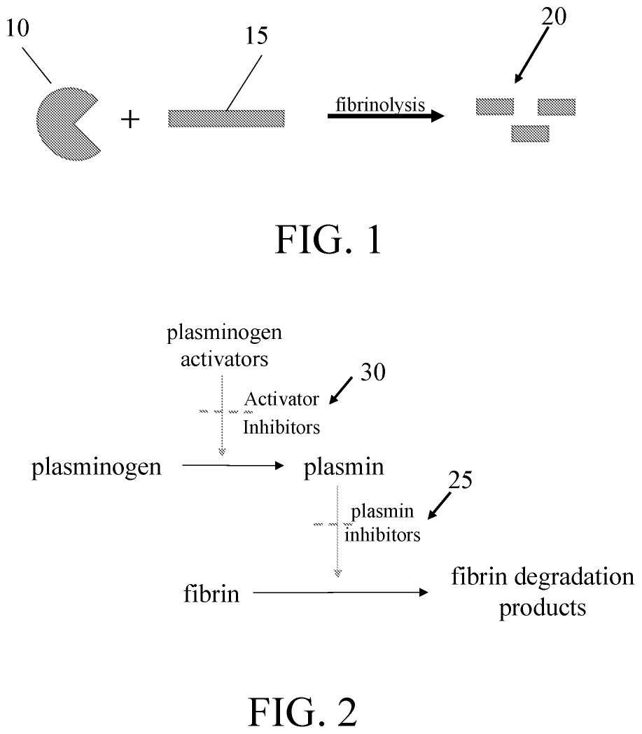

As illustrated in FIG. 1, plasmin 10 is the enzyme that degrades fibrin 15 into fibrin degradation products 20 during fibrinolysis. Fibrinolysis may be regulated by direct (e.g., plasmin inhibitors) or indirect (e.g., activator inhibitors) inhibition of plasmin as shown in FIG. 2. Plasmin may be directly inhibited by serine protease inhibitors (i.e., serpin), such as α₂-antiplasmin and α₂-macroglobin. These inhibitors may bind to the active site of plasmin and prevent the enzymatic cleavage of fibrin. In some embodiments, a sequestering agent may prevent the inhibitory function of direct plasmin inhibitor by specifically binding and/or shielding the inhibitory portion of the direct plasmin inhibitor (e.g., serpins, such as α₂-antiplasmin, α₂-macroglobin). Plasmin may be indirectly regulated (e.g., inhibited) by inhibition of the conversion of plasminogen into plasmin. That is, plasmin may be indirectly inhibited by activator inhibitors as shown in FIG. 2. Plasminogen is a zymogen of plasmin that is enzymatically converted to plasmin by a plasminogen activator, such a tissue plasminogen activator (tPA) and urokinase. Activation of plasminogen may be inhibited by activator inhibitors, such as plasminogen activator inhibitors (e.g., plasminogen activator inhibitor-1, plasminogen activator inhibitor-2), as shown in FIG. 2. In some embodiments, a sequestering agent may specifically bind and/or shield the inhibitory portion of a plasminogen activator inhibitor (PAI).

In some embodiments, the sequestering agent is an exogenous biological molecule. Non-limiting examples of exogenous sequestering agents include plasmin, plasminogen, and serpin inhibitors (e.g., α₂-antiplasmin inhibitors, α₂-macroglobin inhibitors, inhibitors of plasminogen activator inhibitor). In some embodiments, the sequestering agent may be exogenous plasmin and/or plasminogen. The exogenous plasmin may be capable of binding inhibitors, such as α₂-antiplasmin inhibitors and α₂-macroglobin inhibitors, and reducing inhibition of other (e.g., native, exogenous) plasmin in the sample. Exogenous plasminogen may be converted to plasmin and function as described above with respect to exogenous plasmin. In certain embodiments, the sequestering agent is an exogenous serpin inhibitor. For example, the sequestering agent may be a monoclonal antibody that binds and prevents the functioning of α₂-antiplasmin inhibitors. Suitable monoclonal antibody to α₂-antiplasmin are described in Reed et al., “Functional characterization of monoclonal antibody inhibitors of alpha 2-antiplasmin that accelerate fibrinolysis in different animal plasmas.” Hybridoma 1997 June; 16(3):281-6, which is incorporated by reference in its entirety.

It should be understood that exogenous (e.g., with respect to a sequestering agent) refers to the ex-vivo addition of an agent (e.g. sequestering agent) to a sample, wherein the agent being added ex-vivo may be from the same patient for whom the assay is being performed, a different human, or may be from an alternative source, including a non-human source.

In some embodiments, the sequestering agents described herein have a suitable binding affinity to direct and/or indirect plasmin inhibitor. As used herein, “binding affinity” refers to the apparent association constant or K_{A}. The K_{A} is the reciprocal of the dissociation constant (K_{D}). The antibody described herein may have a binding affinity (K_{D}) of at least 10⁻⁵, 10⁻⁶, 10⁻⁷, 10⁻⁸, 10⁻⁹, 10⁻¹⁰ M, or lower. An increased binding affinity corresponds to a decreased K_{D}. Binding affinity can be determined by a variety of methods including equilibrium dialysis, equilibrium binding, gel filtration, ELISA, surface plasmon resonance, or spectroscopy (e.g., using a fluorescence assay).

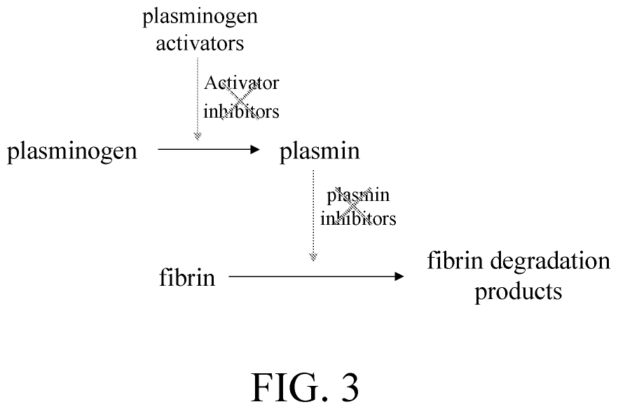

Without being bound by theory, it is believed that the sequestering agents may reduce and/or eliminate downregulation of fibrinolysis by sequestering one or more direct or indirect plasmin inhibitors as illustrated in FIG. 3. It also believed that the sequestering agents (e.g., plasmin and/or plasminogen) may upregulate fibrinolysis by increasing the concentration of plasmin. Regardless of the mechanism, the sequestering agents are believed to alter the level of plasmin (e.g., alter the level of active plasmin, increase plasmin concentration) and/or its inhibitors (e.g., decrease the active inhibitor concentration) in the sample during at least a portion of the assay. The sequestering agents may cause the sample to have levels of plasmin and/or its inhibitors that would not naturally occur in the sample.

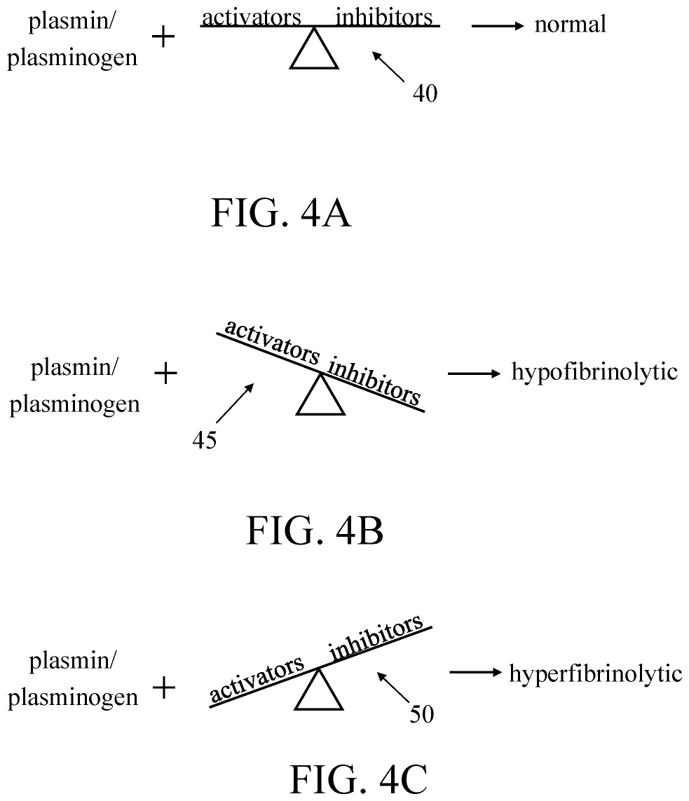

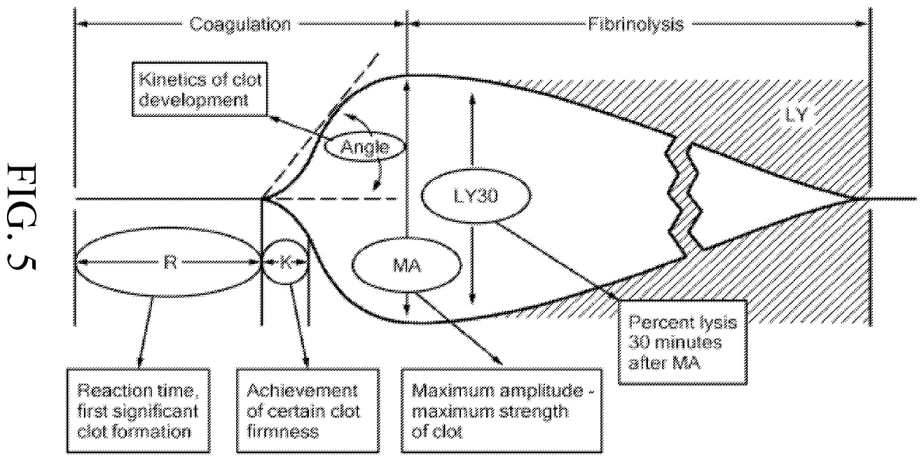

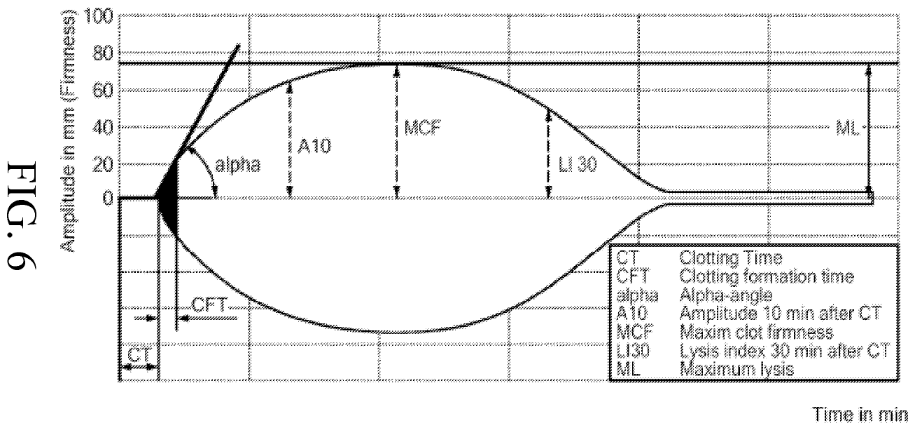

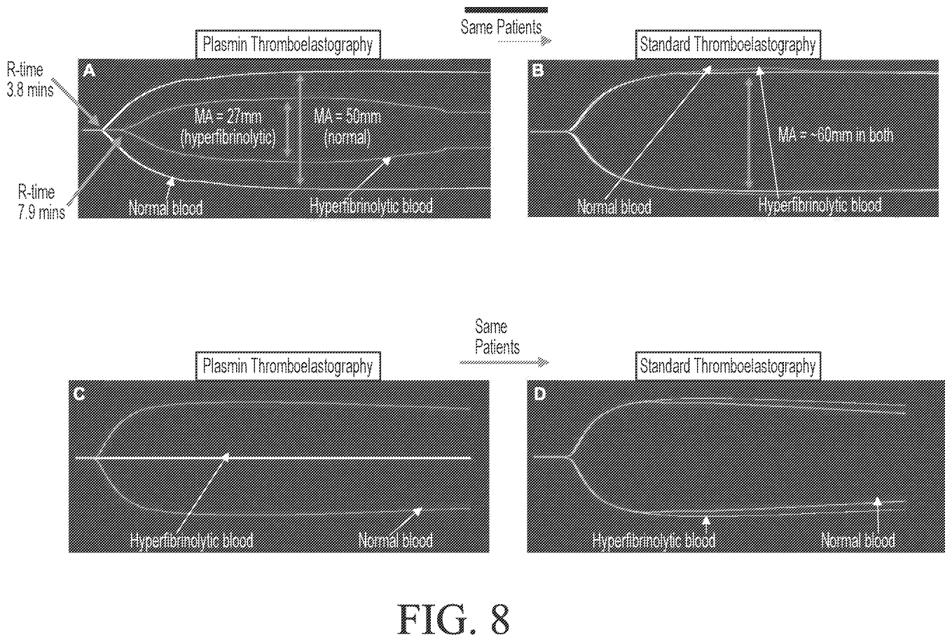

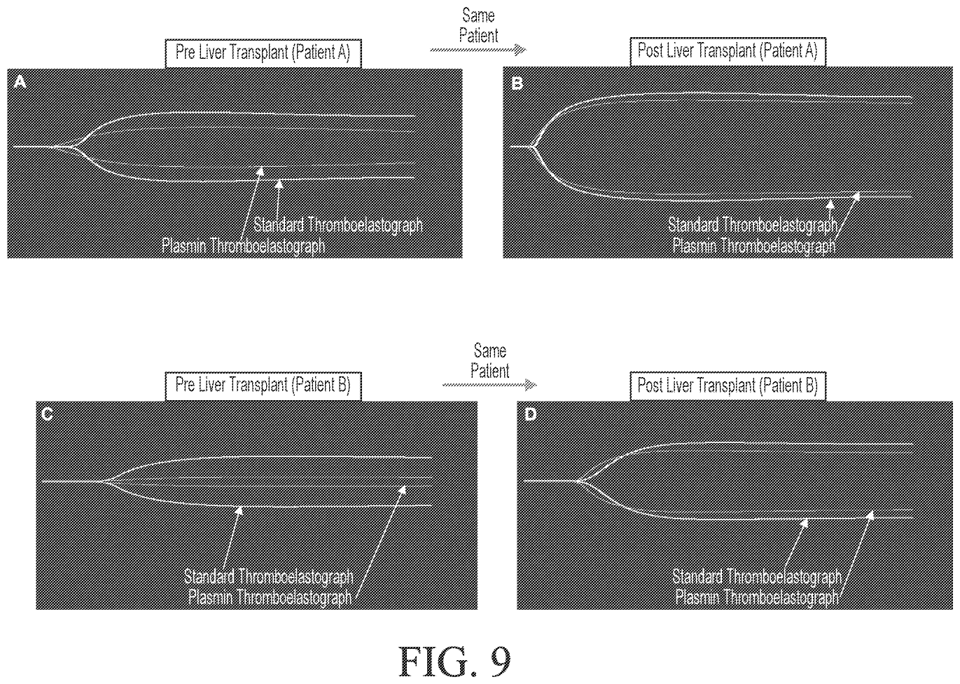

Furthermore, samples with abnormal coagulation and/or fibrinolysis may be less capable of regulating these non-natural levels resulting in significant disruption of coagulation and/or fibrinolysis that is reflected in the assay results (e.g., viscoelastic trace). Accordingly, utilization of sequestering agents may magnify the difference between normal and abnormal samples in a functional assay, as described in more detail below, as shown in FIGS. 8 and 9. For example, as schematically illustrated in FIGS. 4A-4C, utilization of plasmin and/or plasminogen as the sequestering agent may produce assay results that are indicative of an underlying coagulation and/or fibrinolysis phenotype. For instance, as shown in FIG. 4A, combination of plasmin and/or plasminogen with a sample having a normal balance 40 of fibrinolysis activators and inhibitors may result in an assay result (e.g., viscoelastic trace) indicative of a normal phenotype or a predisposition toward a normal phenotype, e.g., upon a triggering event. Conversely, as schematically shown in FIG. 4B, combination of plasmin and/or plasminogen with a sample having an overabundance 45 of fibrinolysis inhibitors may result in an assay result (e.g., viscoelastic trace) indicative of a hypofibrinolysis phenotype or a predisposition toward hypofibrinolysis phenotype (e.g. “fibrinolysis shutdown”), e.g., upon a triggering event. As another example, as schematically shown in FIG. 4C, combination of plasmin and/or plasminogen with a sample having an overabundance 50 of fibrinolysis activators or lack of fibrinolysis inhibitors may result in an assay result (e.g., viscoelastic trace) indicative of a hyperfibrinolytic phenotype or a predisposition toward hyperfibrinolysis phenotype, e.g., upon a triggering event.

In some embodiments, further information regarding a coagulation and/or fibrinolysis abnormality, such as the identification of specific inhibitor and/or activator abnormalities, may be obtained from performing the coagulation and fibrinolysis assays described herein in combination with another assay or component thereof. In some embodiments, the other assay may be a traditional assay, a conventional functional assay, or a functional assay that includes certain activators of coagulation and/or fibrinolysis (e.g., tissue plasminogen activator, urokinase, streptokinase). In certain embodiments, the other assay may be an assay of the present disclosure that utilizes a different sequestering agent (e.g., serpin inhibitor). In some embodiments, the assays may be performed in parallel with or within a relatively short period of time of each other (e.g., less than or equal to 2 hours). In some embodiments, the other assay may be a functional assay that includes one or more activators of fibrinolysis, such as tissue plasminogen activator, urokinase, and/or streptokinase. In some embodiments, the two assays may be combined into a single assay. For instance, the sequestering agent (e.g., plasmin and/or plasminogen) may be combined with an activator of fibrinolysis. In certain embodiments, the upstream activator of fibrinolysis is tissue plasminogen activator.

In certain embodiments, a combination assay may comprise combining one or more exogenous sequestering agent (e.g., plasmin and/or plasminogen) and exogenous tissue plasminogen activator. The assay may further comprise one or more steps, described herein. In some embodiments, the combination assay may allow irregularities in plasminogen activator inhibitors to be determined. In some instances, the concentration of tissue plasminogen activator in the combination assay may be greater than or equal to about 50 ng/ml and less than or equal to about 200 ng/mL (e.g., greater than or equal to about 75 ng/ml and less than or equal to about 150 ng/mL).

In some embodiments, a certain amount of the sequestering agent is combined with the sample. The amount of sequestering agent may influence the assay duration and/or the sensitivity of the assay. In some embodiments, the exogenous plasmin and/or plasminogen concentration in the sample is greater than or equal to about 10%, greater than or equal to about 12%, greater than or equal to about 14%, greater than or equal to about 15%, greater than or equal to about 16%, greater than or equal to about 18%, greater than or equal to about 20%, greater than or equal to about 22%, or greater than or equal to about 24% of the maximum theoretic possible plasmin generation in an average adult human. In some instances, the plasmin and/or plasminogen concentration in the sample is less than or equal to about 40%, less than or equal to about 30%, less than or equal to about 25%, less than or equal to about 24%, less than or equal to about 22%, less than or equal to about 20%, less than or equal to about 18%, less than or equal to about 16%, less than or equal to about 15%, less than or equal to about 14%, or less than or equal to about 12% of the maximum theoretic possible plasmin generation in an average adult human. All combination of the above-referenced ranges are possible (e.g., greater than or equal to about 10% and less than or equal to about 25%). In some embodiments, the plasmin and/or plasminogen concentration in the sample is about 10%, about 20%, or about 25% of the maximum theoretic possible plasmin generation in an average adult human. The maximum plasmin concentration that could be generated in a human may be calculated from the average circulating plasminogen concentration (i.e., 176 μg/mL (2 μM) as described in Robbins KC. The human plasma fibrinolytic system: regulation and control. Mol Cell Biochem. 1978; 20(3):149-157) which is incorporated by reference in its entirety) by taking in to account loss of N-terminal cleavage fragments due to activation.

In some embodiments, the exogenous plasmin and/or plasminogen concentration in the sample is greater than or equal to about 14 μg/mL, greater than or equal to about 16 μg/mL, greater than or equal to about 16.6 μg/mL, greater than or equal to about 18 μg/mL, greater than or equal to about 20 μg/mL, greater than or equal to about 22 μg/mL, greater than or equal to about 24 μg/mL, greater than or equal to about 26 μg/mL, greater than or equal to about 28 μg/mL, greater than or equal to about 30 μg/mL, greater than or equal to about 32 μg/mL, greater than or equal to about 33.2/mL μg/mL, greater than or equal to about 34 μg/mL, greater than or equal to about 36 μg/mL, greater than or equal to about 38 μg/mL, greater than or equal to about 40 μg/mL, or greater than or equal to about 41.5 μg/mL. In some instances, the plasmin and/or plasminogen concentration in the sample is less than or equal to about 60 μg/mL, less than or equal to about 45 μg/mL, less than or equal to about 43 μg/mL, less than or equal to about 41.5 μg/mL, less than or equal to about 40 μg/mL, less than or equal to about 38 μg/mL, less than or equal to about 36 μg/mL, less than or equal to about 34 μg/mL, less than or equal to about 33.2 μg/mL, less than or equal to about 32 μg/mL, less than or equal to about 30 μg/mL, less than or equal to about 28 μg/mL, less than or equal to about 26 μg/mL, less than or equal to about 24 μg/mL, less than or equal to about 22 μg/mL, less than or equal to about 20 μg/mL, or less than or equal to about 18 μg/mL. All combination of the above-referenced ranges are possible (e.g., greater than or equal to about 14 μg/mL and less than or equal to about 45 μg/mL, greater than or equal to about 16 μg/mL and less than or equal to about 43 μg/mL, greater than or equal to about 16.6 μg/mL and less than or equal to about 41.5 μg/mL, greater than or equal to about 33.2 μg/mL and less than or equal to about 41.5 μg/mL, greater than or equal to about 16.6 μg/mL and less than or equal to about 33.2 μg/mL). In a particular embodiment, the plasmin and/or plasminogen concentration in the sample is greater than or equal to about 16.6 μg/mL and less than or equal to about 41.5 μg/mL.

In general, the term “about” means within an acceptable error range for the particular value as determined by one of ordinary skill in the art, which will depend in part on how the value is measured or determined, i.e., the limitations of the measurement system. For example, in regard to concentration, “about” can mean within an acceptable standard deviation, per the practice in the art. “About” can mean a range of up to ±20%, preferably up to ±10%, more preferably up to ±5%, and more preferably still up to ±1% of a given value.

In some embodiments, the duration of the functional assays described herein may be relatively short. For instance in some embodiments, the assay duration may be less than or equal to about 45 minutes, less than or equal to about 40 minutes, less than or equal to about 35 minutes, less than or equal to about 30 minutes, less than or equal to about 25 minutes, less than or equal to about 20 minutes, less than or equal to about 15 minutes, or less than or equal to about 10 minutes. In some embodiments, the assay duration may be greater than or equal to about 5 minutes and less than or equal to about 30 minutes (e.g., greater than or equal to about 5 minutes and less than or equal to about 20 minutes, greater than or equal to about 5 minutes and less than or equal to about 15 minutes, greater than or equal to about 5 minutes and less than or equal to about 10 minutes). In certain embodiments, the assay duration may be less than or equal to about 15 minutes (e.g., less than or equal to about 10 minutes).

As noted above, one or more sequestering agents may be utilized in a functional assay. In general, any suitable functional assay may be used. Non-limiting examples of suitable functional assays include viscoelastic assays, such as thromboelastography (e.g. TEG 5000 or TEG 6s), and rotational thromboelastometry (e.g. ROTEM delta). In some embodiments, the functional assay may be performed on a microfluidic device, such as in the T-TAS® (Fujimori Kogyo Co., LTD) assay. In some embodiments, the functional assay may be performed using a device that use reporters (e.g., fluorescent, absorption) to measure one or more properties of coagulation and/or fibrinolysis of whole blood and/or blood product. In some embodiments, the functional assay may be performed using a device that use optical measurements and metrics, vibration, rheometry, or other methods, such as Hemolyzer devices (Analyticon® Biotechnologies), Sonoclot® analyzers (©Sienco, Inc), Quantra™ (HemoSonics, LLC), and others.

In some embodiments, the functional assay is a viscoelastic assay. In general, viscoelastic assays (e.g., thromboelastography, thromboelastometry, ROTEM®, TEG® and Sonoclot®) measure the change in viscoelastic properties of whole blood at the patient's body temperature during clot formation and dissolution. In general, patient whole blood is added to a heated cup and clot formation is initiated via a clotting activator (e.g., kaolin, calcium, citric acid). A sensor (e.g., pin, torsion wire) is positioned within the cup. During the assay, either the sensor or cup is moved (e.g., via rotation, vibration), yielding movement between the pin and the cup. The force opposing the movement is measured during the assay. When the clotting activator is added, small amounts of thrombin are generated. The thrombin then activates platelets that as a result of activation expose phosphatidylserine. The phosphatidylserine supports assembly of coagulation factor complexes and amplifies and propagates thrombin generation. Once larger amounts of thrombin are formed, thrombin cleaves fibrinogen to form fibrin. The lag time between the start of coagulation and the time for fibrin to start forming is called the reaction time or clot time and is mostly dependent on coagulation factors and hematocrit. The fibrin strands that are forming during this process restrict the movement between the sensor and the cup resulting in a higher force opposing the movement of the sensor or cup. Fibrin lyses begins upon sufficient production of active plasmin. As the fibrin lyses, movement between the pin and the cup becomes unrestricted and is also monitored by the instrument, yielding information on fibrinolysis.

The change in movement during the assay is relayed through the sensor and detected, e.g., optically (ROTEM®) or electronically (TEG®) and converted to a trace reflecting the rate of fibrin formation and fibrinolysis. Numerical results can be derived from the trace that provide information on the time of onset of fibrin formation, the rate of fibrin formation, the strength of the fibrin clot, the amount of fibrin formation, and fibrinolysis at defined endpoints as illustrated in FIGS. 5 and 6. Comparison of viscoelastic traces and/or numerical data to healthy controls allow for the coagulation phenotype (e.g., hypofibrinolytic) and/or fibrinolytic phenotype (e.g., hyperfibrinolytic) of a patient or predisposition toward a phenotype to be determined.

Thus, parameters that may be used to determine a coagulation and/or fibrinolysis disorder using a TEG or TEM assays, include the maximum strength of the clot which is a reflection of clot strength. This is the MA value in the TEG assay, and the MCF value in the TEM assay. The reaction time (R) in TEG (measured in seconds or minutes) and clotting time (CT) in TEM is the time until there is first evidence of clot; clot kinetics (K, measured in minutes) is a parameter in the TEG test indicating the achievement of clot firmness; and a in TEG or alpha-angle in TEM is an angular measurement from a tangent line drawn to the curve of the TEG tracing or TEM tracing starting from the point of clot reaction time that is reflective of the kinetics of clot development.

The schematic shown in FIG. 5 depicts a TEG tracing when fibrinolysis occurs. As shown in FIG. 5, the resulting hemostasis profile (i.e., a TEG tracing curve) is a measure of the time it takes for the first fibrin strand to be formed, the kinetics of clot formation, the strength of the clot (measured in millimeters (mm) and converted to shear elasticity units of dyn/cm²) and dissolution of clot. See also Donahue et al., J. Veterinary Emergency and Critical Care: 15(1): 9-16 (March 2005), herein incorporated by reference. The descriptions for several of these measured parameters of the TEG tracing curve are as follows:

R is the period of time of latency from the time that the blood was placed in the thromboelastography analyzer until the initial fibrin formation. The R range will vary based on the particular TEG assay performed (e.g., type of blood sample being tested (e.g., plasma only or whole blood). For patients in a hypocoagulable state (i.e., a state of decreased coagulability of blood), the R number is longer, while in a hypercoagulable state (i.e., a state of increased coagulability of blood), the R number is shorter. In the methods described herein, the R value (in minutes or seconds) can be used to determine a pathological phenotype of a subject.

K value (measured in minutes) is the time from the end of R until the clot reaches 20 mm and this represents the speed of clot formation. In a hypocoagulable state, the K number is longer, while in a hypercoagulable state, the K number is shorter. In the methods described herein, the K value can be used to determine a pathological phenotype of a subject.

a (alpha) value measures the rapidity of fibrin build-up and cross-linking (clot strengthening). Thus, the a (alpha) value is reflective of the coagulation process. It is angle between the line formed from the split point tangent to the curve and the horizontal axis. In a hypocoagulable state, the a degree is lower, while in a hypercoagulable state, the a degree is higher. In the methods described herein, the a value can be used to determine a pathological phenotype of a subject.

MA or Maximum Amplitude in mm, is a direct function of the maximum dynamic properties of fibrin and platelet bonding and represents the ultimate strength of the blood clot. The MA value is reflective of the coagulation process. If the blood sample tested has a reduced platelet function (e.g., platelet-free plasma), this MA represents the strength of the clot based mainly on fibrin. Decreases in MA may reflect a hypocoagulable state (e.g., with platelet dysfunction or thrombocytopenia), whereas an increased MA (e.g., coupled with decreased R) may be suggestive of a hypercoagulable state.

LY30 is a measure of amplitude reduction a fixed time (e.g., 30 minutes) after MA and represents clot retraction, or lysis. The LY30 value is thus a percentage decrease in amplitude a fixed time (e.g., 30 minutes) after the Ma, and is reflective of the fibrinolysis process. The larger the LY30 value, the faster fibrinolysis occurs. When no fibrinolysis occurs, the amplitude value at the MA tracing stays constant or may decrease slightly due to clot retraction. However, as fibrinolysis occurs (e.g., in a healthy individual), the curve of the TEG tracing starts to decay. The resultant loss in potential area-under-the-curve in the fixed time (e.g., 30 minutes) following Maximum Amplitude in the TEG assay is called the LY30 (see FIG. 5). LY30, the percentage of lysis a fixed time (e.g., 30 minutes) after the maximum amplitude point (expressed as a percentage of the clot lysed) indicates the rate of fibrinolysis. In some embodiments, clot firmness (G, measured in dynes/cm²) may be used to express LY30.

The schematic shown in FIG. 6 depicts a ROTEM tracing when fibrinolysis occurs. As shown in FIG. 6, the resulting hemostasis profile (i.e., a TEG tracing curve) is a measure of the time it takes for the first fibrin strand to be formed, the kinetics of clot formation, the strength of the clot and dissolution of clot. The descriptions for several of these measured parameters of the ROTEM tracing curve are as follows:

CT (clotting time) is the period of time of latency from the time that the blood was placed in the ROTEM analyzer until the clot begins to form. This CT time can be used to determine a pathological phenotype of a subject.

CFT (Clot formation time): the time from CT until a clot firmness of 20 mm point has been reached. This CFT time can be used to determine a pathological phenotype of a subject.

alpha-angle: The alpha angle is the angle of tangent at 2 mm amplitude. This alpha angle can be used to determine a pathological phenotype of a subject.

MCF (Maximum clot firmness): MCF is the greatest vertical amplitude of the trace. MCF reflects the absolute strength of the fibrin and platelet clot. If the blood sample tested has a reduced platelet function, this MCF is a function of mainly the fibrin bonding strength. The MCF value can be used to determine a pathological phenotype of a subject.

A10 (or A5, A15 or A20 value). This value describes the clot firmness (or amplitude) obtained after 10 (or 5 or 15 or 20) minutes and provide a forecast on the expected MCF value at an early stage. Any of these A values (e.g., A10) can be used to determine a pathological phenotype of a subject.

LI 30 (Lysis Index after 30 minutes). The LI30 value is the percentage of remaining clot stability in relation to the MCF value at 30 min after CT. This LI30 value may be used as a non-limiting coagulation characteristic value in accordance with the methods described herein. When no fibrinolysis occurs, the amplitude value at the MCF on a TEM tracing stays constant or may decrease slightly due to clot retraction. However, as fibrinolysis occurs (e.g., in a hypocoagulable state), the curve of the TEM tracing starts to decay. LI30 corresponds to the LY30 value from a TEG tracing. ML (Maximum Lysis). The ML parameter describes the percentage of lost clot stability (relative to MCF, in %) viewed at any selected time point or when the test has been stopped. This ML value can be used to determine a pathological phenotype of a subject.

A low LI 30 value or a high ML value indicates hyperfibrinolysis. While in normal blood fibrinolysis activity is quite low, in clinical samples a more rapid loss of clot stability by hyperfibrinolysis may lead to bleeding complications which can be treated by the administration of a therapeutic agent that strengthens a blood clot or slows the dissolution of a blood clot, such as an antifibrinolytic agent.

It should be understood that, in some embodiments, the assay duration of assays described herein may be less than the conventional time indicated for measurement of certain assay readout features (e.g., fibrinolysis features such as LY30). In some such cases, surrogates for these assay readout features that provide substantially the same or more information (e.g., diagnostic and/or prognostic value) may be used. For instance, R-time relative to the healthy control standard may be used as a surrogate for LY30. For example, a prolonged R-time relative the healthy control standard for the assay described herein may be indicative of a hyperfibrinolytic phenotype or a predisposition toward a hyperfibrinolytic phenotype, e.g., upon a triggering event. In some instances, maximum amplitude relative to the healthy control standard may be used as a surrogate. For example, a reduced maximum amplitude relative to the healthy control standard may be indicative of a hyperfibrinolytic phenotype or a predisposition toward a hyperfibrinolytic phenotype, e.g., upon a triggering event. As another example, a reduced angle relative to the healthy control standard may be used as a surrogate. A reduced angle relative to the healthy control standard may be indicative of a hyperfibrinolytic phenotype or a predisposition toward a hyperfibrinolytic phenotype, e.g., upon a triggering event. In some embodiments, R-time, maximum amplitude, time to maximum amplitude, and/or angle may be used as surrogates for certain conventional assay readouts (e.g., associated with fibrinolysis, LY30) with substantially the same and/or improved accuracy and sensitivity. It should also be understood that though the above refers to TEG, the description applies to similar or equivalent features in other assays (e.g., ROTEM). For instance, clotting time, maximum clot firmness, alpha-angle, and/or time to maximum clot firmness may be used as surrogates for certain conventional assay readouts (e.g., associated with fibrinolysis, LI 30) with substantially the same and/or improved improved accuracy, specificity, sensitivity, diagnostic value, and/or prognostic value.

In general, the assay results of a test sample may be compared to a healthy control as well as a blank control. The blank control is not a sample from a subject. In some embodiments, the blank control may comprise one or more proteins to control for contact activation. In certain embodiments, the blank control may comprise lyophilized albumin, milk proteins, and/or plant-based “milk” substitute proteins.

In general, the assay may be performed at any suitable temperature. In certain embodiments, the assay may be performed at physiologically relevant temperatures. For instance, the assay be performed at the subject's body temperature (e.g., greater than or equal to about 36° C. and about 38° C.). In certain embodiments, the assay may be performed at a temperature of greater than or equal to about 0° C. and about 60° C. (e.g., greater than or equal to about 0° C. and about 55° C., greater than or equal to about 20° C. and about 40° C.).

In some embodiments, after combination of the sequestering agent (e.g., plasmin and/or plasminogen) with the sample, the resulting assay mixture may be allowed to incubate for a relatively short period of time. For instance, in some embodiments, the incubation time may be less than or equal to about 10 minutes, less than or equal to about 8 minutes, less than or equal to about 5 minutes, or less than or equal to about 2 minutes.

In some embodiments, the exogenous sequestering agent (e.g., plasmin and/or plasminogen) is combined with the sample according to standard laboratory and/or clinical procedures. For instance, in some embodiments, the sample (e.g., blood) is collected in a container (e.g., blood collection container). In some embodiments, the container comprises one or more exogenous sequestering agents (e.g., exogenous plasmin and/or plasminogen). For example, the exogenous sequestering agent may be present in the container in solid (e.g., lyophilized) form. As another example, the exogenous sequestering agent may be present in the container in liquid. Regardless of the form of the exogenous sequestering agent, the sample may be added to a container comprising the sequestering agent. In certain embodiments, one or more exogenous sequestering agents (e.g., exogenous plasmin and/or plasminogen) may be added to a container comprising a sample (e.g., whole blood, plasma, serum, platelets, red blood cells). In some embodiments, the sequestering agent is be added to the sample in solid form. In other embodiments, the sequestering agent is be added to the sample in liquid form.

In general, the exogenous sequestering agents (e.g., plasmin and/or plasminogen) may be relative pure. In some embodiments, the purity of the exogenous sequestering agents (e.g., plasmin and/or plasminogen) may be greater than or equal to about 90%, greater than or equal to about 90%, greater than or equal to about 92%, greater than or equal to about 94%, greater than or equal to about 95%, greater than or equal to about 96%, greater than or equal to about 97%, greater than or equal to about 98%, or greater than or equal to about 99%.

In general, the sample is blood or a blood product (e.g., whole blood, plasma, serum, platelets, red blood cells, treated serum), collectively referred to as a blood or blood-derived sample. In some embodiments, the blood product is whole blood, plasma, serum, platelets, red blood cells, and/or platelet poor plasma with or without anticoagulant(s). In certain embodiments, the sample is whole blood.

A coagulation and fibrinolysis assay kit may include a sample collection chamber and exogenous sequestering agent (e.g., plasmin and/or plasminogen). The sample collection chamber may be free of the subject sample and/or may be configured to contain the subject sample. The kit may also include any solvents, solutions, buffer agents, acids, bases, salts, additives, etc. needed for the assay. Different kits may be available for different sequestering agents (e.g., plasmin, plasminogen, serpin inhibitors) and/or for combination assays. The kit may also include instructions on how to use the materials in the kit. The subject sample is typically provided by the user of the kit.

The coagulation and fibrinolysis assay described herein may be used for a wide variety of clinical applications. In some embodiments, the coagulation and fibrinolysis assay described herein may be used in the treatment and/or diagnosis of cardiovascular disease (e.g. myocardial infarction, cerebrovascular accident), cancer, traumatic injury, liver disease, pre- and post-organ transplant, obstetrics, gynecology, end-stage renal disease, hemodialysis, and diseases requiring or potentially requiring any surgical intervention where bleeding or clotting is a risk, as well as general medical screening and wellness evaluations (e.g. routine physical examinations).

In some embodiments, the sequestering agent is exogenous plasmin and/or plasminogen. Particular embodiments, in which the sequestering agent is exogenous plasmin and/or plasminogen are described below. For ease of reference throughout the disclosure below in which the sequestering agent is exogenous plasmin and/or plasminogen, embodiments of the inventive tests and assays disclosed are collectively referred to as a “Plasmin/Plasminogen Coagulation and Fibrinolysis Assay” (“PCFA”). In certain embodiments, a PCFA can identify patients at high-risk for severe bleeding and death from too much fibrinolysis, providing answers in, for example, 10-15 minutes that can be used by clinicians to make life-saving, time-dependent, personalized medical decisions regarding the administration of anti-fibrinolytic therapies and/or specific blood products targeted at a patient's specific pathologic abnormality. In certain embodiments, a PCFA can identify patients at high-risk for organ failure and thrombotic complications (e.g. deep venous thrombosis, pulmonary embolism, etc.) due to inability to break down blood clots to maintain vascular patency, providing answers in, for example, 60-90 minutes that can be used by clinicians to make personalized medical decisions regarding the administration of anti-coagulant or other therapies targeted at a patient's specific pathologic abnormality. For ease of reference throughout the remainder of this disclosure, hyperfibrinolysis (severe bleeding risk) and fibrinolysis shutdown (severe risk of organ failure and thrombotic complications) may be collectively referred to as “pathologic fibrinolysis phenotypes.”

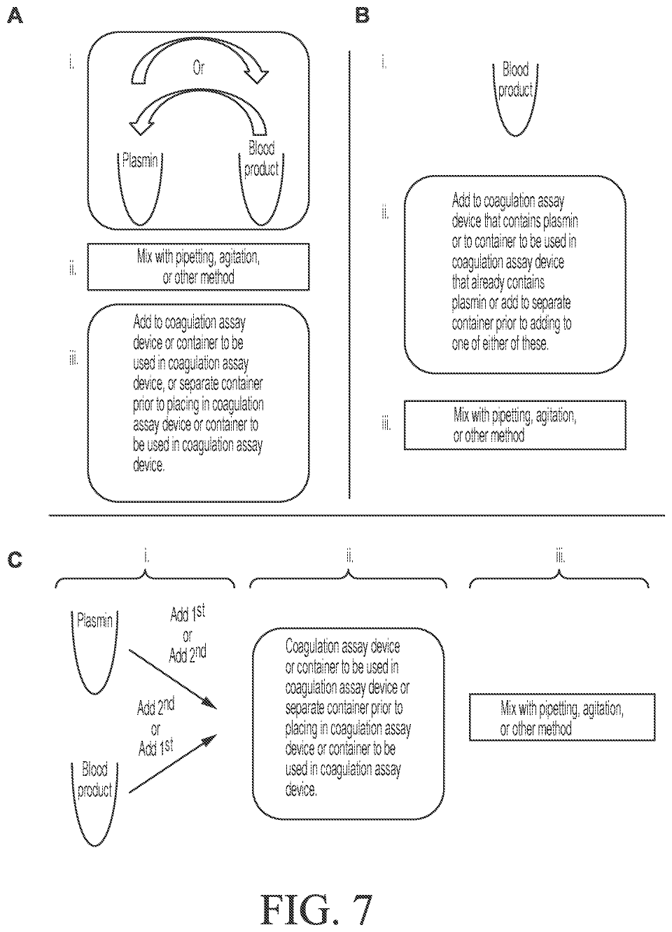

A schematic of the coagulation and/or fibrinolysis assay is plasmin and/or plasminogen is shown in FIG. 7. Panel A in FIG. 7 demonstrates one embodiment of combining plasmin with blood/blood product(s) prior to use in a functional coagulation assay device, where other embodiments such as those described in the text may be performed including using plasminogen instead of (or in addition to) plasmin, incubations at various temperatures, pH titrations, addition of anti-coagulants and/or reversal agents for anti-coagulants, addition of coagulation activators, addition of additional fibrinolytic agents or proteins, and/or addition of fibrinolysis inhibitor agents or proteins. Albumin or milk product (lyophilized or in solution vehicle) can be used in the same way as plasmin/plasminogen to run control PCFA assays for comparison. Panel B demonstrates another embodiment for performing PCFA, where again albumin or milk product (lyophilized or in solution vehicle) can be used in the same way as plasmin/plasminogen to run control PCFA assays for comparison. Panel C demonstrates another embodiment for performing PCFA, where again albumin or milk product (lyophilized or in solution vehicle) can be used in the same way as plasmin/plasminogen to run control PCFA assays for comparison. The term “Plasmin” in the diagram may represent plasmin and/or plasminogen, depending on the embodiment.

In certain embodiments of the PCFA, human or other mammalian blood is collected into blood collection tubes according to standard/common clinical practice to which exogenous plasmin and/or plasminogen may be added, or, in other embodiments, the blood is collected in a novel fashion directly into a tube containing plasmin and/or plasminogen. In certain embodiments the blood collection tubes can be used without anticoagulant or, in other embodiments, may contain an anticoagulant (such as citrate, heparin, low molecular weight heparins, synthetic pentasaccharides, EDTA, other Calcium chelating agents, thrombin inhibitors, or others). In certain embodiments, PCFA may be performed on whole blood, anticoagulated whole blood, and/or blood products derived from whole blood (e.g. plasma, anticoagulated plasma, platelet-poor plasma, anticoagulated platelet-poor plasma, cryoprecipitate, fibrinogen, etc.) collected in blood collection tubes that either did or did not contain plasmin and/or plasminogen, a combination of any of these, or a combination of any of these that also includes exogenous synthetic and/or recombinant blood products (e.g. addition of recombinant Tissue Factor to a PCFA assay performed on whole blood). The blood or blood product of choice is then mixed exogenously with the enzyme plasmin and/or plasminogen if the blood or blood product was not already collected in a plasmin/plasminogen-containing blood collection tube or if the blood or blood product was collected or contained in a blood collection tube that did not contain enough plasmin and/or plasminogen to reach the desired plasmin/plasminogen concentration. In general, where the term “exogenous” is used herein, it should be understood that this means the ex-vivo addition of an agent (e.g. plasmin and/or plasminogen) to blood or the chosen blood product, where the agent being added ex-vivo may be from the same patient for whom the PCFA test is being performed, or may be from an alternative source to include a non-human source of plasmin/plasminogen. In certain embodiments this involves adding the blood or blood product of choice to lyophilized plasmin and/or plasminogen, or collecting the blood or blood product of choice in a tube containing lyophilized plasmin and/or plasminogen, or collecting the blood or blood product of choice in a blood collection tube containing lyophilized plasmin and/or plasminogen. In certain embodiments this involves adding the blood or blood product of choice to a solution containing plasmin and/or plasminogen, or collecting the blood or blood product of choice in a tube containing a solution that contains plasmin and/or plasminogen, or collecting the blood or blood product of choice in a blood collection tube containing a solution that contains plasmin and/or plasminogen. In certain embodiments this involves adding lyophilized plasmin and/or plasminogen to the blood or blood product of choice. In certain embodiments this involves adding a solution containing plasmin and/or plasminogen to the blood or blood product of choice.

In certain embodiments the lyophilized plasmin and/or plasminogen may contain albumin, while in other embodiments a solution containing plasmin and/or plasminogen may contain albumin. In certain embodiments the lyophilized plasmin and/or plasminogen may contain animal milk or milk products, while in other certain embodiments a solution containing plasmin and/or plasminogen may contain animal milk or milk products. In certain embodiments the lyophilized plasmin and/or plasminogen may contain plant-based “milk” substitutes (e.g. from soybean-bean based “milk” substitutes), while in other certain embodiments a solution containing plasmin and/or plasminogen may contain plant-based milk substitutes (e.g. from soybean-based “milk” substitutes). In certain embodiments utilizing plasmin and/or plasminogen, the plasmin and/or plasminogen used can be recombinant, while in other embodiments plasmin can be made and purified from recombinant plasminogen. In certain embodiments the plasmin and/or plasminogen used can be isolated from blood or blood products. In certain embodiments utilizing plasmin, the plasmin used can be isolated plasmin that is made from plasminogen isolated from blood or blood products. In certain embodiments the plasmin and/or plasminogen may be missing part or all of its Heavy (A) Chain to include missing any or all of its Kringle Domains and/or Pan-Apple Domain. In certain embodiments the plasmin and/or plasminogen Heavy (A) Chain may contain amino acid substitutions or peptide truncations. In certain embodiments the plasmin and/or plasminogen Light (B) Chain may contain amino acid substitutions or peptide truncations. In certain embodiments the plasmin and/or plasminogen can have post-translational modifications including increased or decreased glycosylation. In certain embodiments a recombinant plasmin-like protein and/or a recombinant plasminogen-like protein may be used that has similar activity as the native protein (e.g. in the case of a recombinant plasmin-like protein, with similar peptidase activity and cleavage specificity as plasmin). In general, where the term “plasmin” or “plasminogen” is used herein, it should be understood that in certain embodiments, a plasmin-like protein or plasminogen-like protein meeting criteria required for the PCFA may be used instead of or in addition to the plasmin/plasminogen.

The final concentration of plasmin and/or plasminogen used in PCFA after it has been mixed with the blood or blood product of choice can range from as low as 0.1 ug/mL in certain embodiments to as high as 200 ug/mL in other certain embodiments, and may fall anywhere between 0.1 ug/mL to 200 ug/mL final concentration depending on the embodiment. In particular embodiments, the final plasmin and/or plasminogen concentration is between 5 and 50 ug/mL. A titration of increasing or decreasing plasmin and/or plasminogen concentrations may also be used in serial or concomitant PCFA assays in certain embodiments in addition to performing and comparing with a non-plasmin/non-plasminogen containing assay for comparison purposes. In certain embodiments, the non-plasmin/non-plasminogen containing comparison assay may be performed with non-anticoagulated or anticoagulated blood or blood product of choice that was collected in a tube or placed in a tube, or placed in an assay tube, or placed in an assay device container, or placed in an assay device containing all the same additives and/or reagents as the tube used for PCFA, inclusive of carrier proteins/protective agents such as albumin and/or milk and/or milk products and/or plant-based “milk” substitutes, and all other steps of the process remaining identical with exception of the presence of the plasmin and/or plasminogen. The volume of blood or blood product of choice mixed with plasmin and/or plasminogen can range from 1 microliter to 100 milliliters, depending on the embodiment. In certain embodiments, the volume of blood or blood product of choice mixed with plasmin and/or plasminogen ranges between 25 microliters and 10 milliliters. In certain embodiments an anti-coagulation reversal agent (e.g. Calcium-containing product, or protamine, or heparin-binding agent, etc.) may be added to the blood or blood product of choice prior to being mixed with plasmin and/or plasminogen, while in other certain embodiments an anti-coagulation reversal agent may be added to the blood or blood product of choice any time after it has been mixed with plasmin and/or plasminogen to include during PCFA performance.

Once plasmin and/or plasminogen has been mixed with blood or the blood product of choice it may be used immediately in PCFA in certain embodiments, while in other certain embodiments it may be incubated for periods of time between 0 and 120 minutes at a temperature between 0 degrees Celsius and 55 degrees Celsius prior to use in PCFA. In some such embodiments, the incubation time is between 0 and 60 minutes. In certain embodiments, the temperature of incubation is between 30 and 40 degrees Celsius. In some embodiments, the temperature during performance of PCFA is between 30 and 40 degrees Celsius. In certain embodiments the pH of the blood or chosen blood product or blood-plasmin and/or blood-plasminogen mixture or chosen blood product-plasmin and/or blood product-plasminogen mixture may be unaltered, while in certain other embodiments the pH may be titrated with an acid, base, or buffer solution to a pH between 5.0 and 10.0, for example a pH is between 6.4 and 8.4. PCFA can then be performed in certain embodiments by adding the blood-plasmin and/or blood-plasminogen mixture or chosen blood product-plasmin and/or blood product-plasminogen mixture (referred to below as the “test sample”) directly to the coagulation assay device of choice, while in other embodiments of PCFA such test sample may be added to a container designed for use in the coagulation assay device. In certain embodiments of PCFA a reversal agent for an anticoagulant is added to the test sample prior to starting PCFA. In certain embodiments of PCFA a reversal agent for an anticoagulant is added to a container that at some point will contain the test sample either prior to or during PCFA. In certain embodiments an anticoagulant reversal agent is added to the test sample after PCFA has started. In certain embodiments an agent may be mixed with the test sample to activate coagulation prior to starting PCFA (e.g. tissue factor, kaolin, thrombin, or other activating agent or protein), while in other certain embodiments an agent may be added to the test sample to activate coagulation while starting PCFA or after starting PCFA (e.g. tissue factor, kaolin, thrombin, or other activating agent or protein).

In certain embodiments PCFA may be performed on a viscoelastic assay device, including but not limited to thromboelastography or rotational thromboelastometry devices, that measure parameters of whole blood and/or blood product coagulation, clot formation, clot strength, and/or fibrinolysis/clot lysis. In certain embodiments PCFA may be performed in microfluidic devices that measure parameters of whole blood and/or blood product coagulation, clot formation, clot strength, and/or fibrinolysis/clot lysis. In certain embodiments PCFA may be performed in other coagulation testing platforms that measure parameters of whole blood and/or blood product coagulation, clot formation, clot strength, and/or fibrinolysis/clot lysis. In certain embodiments PCFA will be performed around the same time as a non-PCFA coagulation test that does not contain plasmin and/or plasminogen

In certain embodiments, coagulation and fibrinolysis assay compositions and assay kits are disclosed. In certain such embodiments, the compositions and assay kits include plasmin and/or plasminogen that is exogenous with respect to the sample being tested. In certain cases, the kits include a sample collection container free of a subject sample for testing and configured to contain such sample, which container also contains an exogenous plasmin and/or plasminogen. In certain embodiments, the assay compositions or kits may be used in to perform a viscoelastic assay, optionally a thromboelastography (TEG) based viscoelastic assay. In other embodiments, the assay is a rotational thromboelastometry-based assay. In certain embodiments, the assay uses a microfluidic device. In certain embodiments, the assay composition or kit may include a plasmin that is a purified and enzymatically active plasmin and/or a plasminogen that is a purified plasminogen that yields enzymatically active plasmin. In certain such embodiments, the purified plasmin is a recombinant plasmin that has greater than 40% amino acid sequence identity to a naturally occurring plasmin, or has greater than 60%, greater than 80%, or greater than 90% amino acid sequence identity to a naturally occurring plasmin. In certain embodiments, the assay compositions or kits may include a plasminogen that is a purified plasminogen that yields enzymatically active plasmin that may in certain embodiments be a recombinant plasminogen that has greater than 40% amino acid sequence identity to a naturally occurring plasminogen, or greater than 60%, greater than 80%, or greater than 90% amino acid sequence identity to a naturally occurring plasminogen. In certain embodiments, the assay compositions or kits include plasmin that is a mammalian, for example human, plasmin and/or plasminogen that is a mammalian, for example human, plasminogen.

Also disclosed are methods comprising combining a blood or blood-derive sample from a patient being tested with a plasmin and/or plasminogen that is exogenous with respect to the blood or blood-derive sample, and performing a coagulation and fibrinolysis assay on the sample. In certain embodiments, the assay of the method performed is a viscoelastic assay, optionally a thromboelastography (TEG) based viscoelastic assay. In certain embodiments, the assay is a rotational thromboelastometry based assay. In certain embodiments, the assay employs a microfluidic device. In yet other embodiments, the assay measures functional coagulation other than through viscoelastic or microfluidic means. In certain embodiments of the methods, the sample from the patient comprises blood from the patient and the plasmin and/or plasminogen is added to a blood collection vial prior to adding the blood from the patient, or is added to the blood collection vial after adding the blood from the patient. In certain embodiments, the assay is performed on a patient sample from a patient with known or suspected liver disease or a patient who has been or is in consideration for being listed for liver transplantation by their medical team, or is undergoing liver transplantation, or is less than one week post liver transplantation. In certain embodiments, the assay is performed on a patient sample from a trauma patient within one month of the initial trauma, and in certain cases within 24 hours of the initial trauma. In certain embodiments, results from the assay are analyzed within 90 minutes of initiating the assay and optionally are continuously analyzed for additional information for up to 120 minutes thereafter to provide information regarding the coagulation state of the patient. In certain embodiments, the assay results are analyzed within 15 minutes of initiating the assay and optionally are continuously analyzed for additional information up to 120 minutes thereafter to provide information regarding the coagulation state of the patient. In certain embodiments, the assay measures a narrowing of a graphical output of a viscoelastic assay (thromboelastography or rotational thromboelastometry), where a signal trending towards the horizontal midline is indicative of enhanced fibrinolysis. In certain embodiments, the assay measures a narrowing of the graphical output of the viscoelastic assay (thromboelastography or rotational thromboelastometry), where a signal trending towards the horizontal midline to a lesser degree than normal is indicative of sub-normal amounts of fibrinolysis (fibrinolysis shutdown), and wherein the assay when performed on a sample from a patient in fibrinolysis shutdown includes exogenous plasminogen results in a signal trending towards the horizontal midline to a greater degree than for a sample from the same patient but not including the exogenous plasminogen indicates the fibrinolysis shutdown is due to plasminogen depletion. In certain embodiments, the methods are performed on a patient that is a human patient.

The graphic illustration shown in FIG. 7 demonstrates non-limiting and non-inclusive methods of use in certain embodiments to help delineate how PCFA can be performed. As shown in FIG. 8, rapid differences between normal and hyperfibrinolytic patients are discernable within 10 minutes using certain embodiments of PCFA (where exogenous plasmin was added), with additional diagnostic differences becoming apparent over the next several minutes. The differences seen in certain embodiments of PCFA compared to non-PCFA tests in hyperfibrinolytic states can include any (or all) of the following: altered time to initial clot formation relative to non-PCFA, reduced rate and/or amount of fibrin polymerization relative to non-PCFA, reduced clot strength relative to non-PCFA, and/or increased fibrinolysis/clot lysis relative to non-PCFA. These changes observed in hyperfibrinolytic states in PCFA relative to non-PCFA testing can be quantified with all the usual metrics of clot formation, fibrin polymerization/fibrinogen function, clot strength, and fibrinolysis/clot lysis that the coagulation assay device of choice normally reports. When a hyperfibrinolytic state is not present, PCFA tests mostly resemble non-PCFA tests done on blood or blood products from the same source (e.g. the same patient), while certain embodiments of PCFA tests demonstrate profoundly different measurement parameters relative to non-PCFA tests when hyperfibrinolyis is present. These differences allow for the sensitive diagnosis of hyperfibrinolytic states when they are present. FIG. 9 demonstrates the powerful diagnostic ability of certain embodiments of PCFA in diagnosing hyperfibrinolytic states in two example patients with severe liver disease immediately prior to undergoing liver transplantation, with marked improvement in their PCFA assay parameters on repeat PCFA assays after being transplanted with healthy livers (i.e. the post-liver transplant PCFA tests much more closely resemble their corresponding non-PCFA tests than they did pre-liver transplant, demonstrating the profound sensitivity at diagnosing hyperfibrinolysis). There is no known test in existence that has the capability that certain embodiments of PCFA does to diagnose pathologic hyperfibrinolytic states in such a short time period, which is critical to helping with clinical decision making and personalized care decisions of sick patients who would very likely benefit from anti-fibrinolytic therapy and/or targeted blood product transfusion therapy; thus information provided by certain embodiments of PCFA can facilitate providing improved and more personalized care decisions for patients with pathological hyperfibrinolysis.

In certain embodiments of PCFA (where exogenous plasminogen is added), as compared to non-PCFA tests, the differences observed in patients who have fibrinolysis shutdown can include any (or all) of the following: increased amounts of lysis in PCFA tests relative to non-PCFA tests (where the PCFA test more closely resembles the normal lysis seen in healthy reference patients), altered time to initial clot formation relative to non-PCFA, altered rate and/or amount of fibrin polymerization relative to non-PCFA, and altered clot strength relative to non-PCFA. Patients with fibrinolysis shutdown who have correction of their measures of fibrinolysis activity back towards reference range (i.e. healthy patients) on certain PCFA tests may benefit from treatment with blood products that contain plasminogen or from anti-coagulation therapy; thus information provided by certain embodiments of PCFA can facilitate providing improved and more personalized care decisions for patients with fibrinolysis shutdown in addition to hyperfibrinolysis states.

One embodiment of the invention involves containers or vials containing lyophilized plasmin and/or plasminogen (with or without a carrier protein(s)/protective agent(s)) or plasmin and/or plasminogen solution (with or without a carrier protein(s)/protective agent(s)) into which the initial blood or blood product of choice is mixed or drawn in to directly as detailed in the text above. Certain embodiments of the invention involve using exogenous plasmin and/or exogenous plasminogen or exogenous plasmin-like protein and/or exogenous plasminogen-like protein, both recombinant and/or native, to unmask pathological fibrinolysis phenotypes using viscolelasic assays including thromboelastography and rotational thromboelastometry, microfluidic devices, and all other functional coagulation assays that provide information on blood clotting and fibrinolysis.

An antibody (interchangeably used in plural form) is an immunoglobulin molecule capable of specific binding to a target, such as an inhibitor of plasmin, through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. The antibodies described herein can be murine, rat, human, or any other origin (including chimeric or humanized antibodies). Antibodies capable of binding plasmin inhibitors can be made by any method known in the art and/or are commercially available. See, for example, Harlow and Lane, (1988) Antibodies: A Laboratory Manual, Cold Spring Harbor Laboratory, New York.

A “subject” includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals, such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female at any stage of development. The animal may be a transgenic animal or genetically engineered animal. In certain embodiments, the subject is non-human animal. In certain embodiments, the animal is fish. A “patient” refers to a human subject in need of treatment of a disease.

“As used herein, the terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The following examples are intended to illustrate certain embodiments of the present invention, but do not exemplify the full scope of the invention.

### Example 1

One major advantage achievable with certain embodiments is improved rapidity and robustness of the assay results, and broader utility for diagnosing a wide variety of hyperfibrinolytic states than typical conventional assays. In other certain embodiments, a major advantage is identifying a correctable cause of fibrinolysis shutdown. This new technology has the opportunity to save tens-of-thousands of lives per year in the U.S. alone, with potential broader global and military impact on acute trauma care, the number one cause of loss of productivity in the world's population. Additional opportunities exist to extend the use of PCFA beyond trauma to include transplant surgery, acute heart disease, stroke, sepsis, and even ambulatory settings such as in cancer patients who have significant alterations in coagulation and fibrinolytic function.

Standard functional clotting and coagulation assays that are amenable to use as a PCFA platform are already FDA approved and are becoming increasingly available in hospitals throughout the United States and Europe. Graphic illustrations demonstrate the exemplary methods of PCFA use in FIG. 7 and described in this example.

In this example, blood samples were first collected via venous blood draw and stored in 3.5-mL tubes containing 3.2% citrate as an anticoagulant. 500 μL of this anticoagulated blood was then pipetted into an Eppendorf® tube or other appropriate container and mixed by gentle inversion. The appropriate amount of a solution of plasmin (i.e., 5 uL of a 1.66 mg/mL plasmin solution) versus vehicle control was added to the 500 uL aliquot of blood to get to the desired concentration and was mixed by gentle inversion. Alternatively lyophilized plasmin in the desired amount may be pre-stored in the tube and the blood can be transferred into the tube. A 340-μL aliquot of the mixture was transferred to a 37° C. TEG cup preloaded with 20 μL of 0.2 mol/L CaCl₂ and the assay was run per the usual manufacturer instructions. This may be done on any patient with possible or suspected changes in their fibrinolysis pathway, as described elsewhere in this application.

FIG. 8 demonstrates the rapid and actionable information provided by this Example of the novel PCFA test when used in thromboelastography at discriminating normal blood from hyperfibrinolytic blood that is at very high risk for hemorrhage and death of the patient, where differences are clear in less than 10 minutes. The patient with the thin line in Panel A and the patient with the bold in Panel C should both receive an anti-fibrinolytic drug such as tranexamic acid immediately and may require targeted blood product transfusion as well. Compare this to the standard (non-PCFA) thromboelastographs (Panel B and Panel D, respectively) of the same patient blood samples that demonstrate no meaningful difference between healthy and hyperfibrinolytic blood in either case even after an hour has passed, leaving the physician to make their best guess as to whether or not anti-fibrinolytic therapy should be given to these patients. An incorrect diagnosis followed by subsequent inappropriate treatment could be fatal in these situations. FIG. 9 demonstrates the powerful diagnostic ability of an exemplary embodiment of PCFA in diagnosing hyperfibrinolytic states in two example patients with severe liver disease immediately prior to undergoing liver transplantation, with marked improvement in their PCFA assay parameters on repeat PCFA assay after being transplanted with a healthy liver.

Panel A in FIG. 8 shows the difference between normal blood (bold line) and profoundly pathologic hyperfibrinolytic blood (thin line) at very high risk of hemorrhage and death from bleeding, discriminating in under 10 minutes whether or not a patient should be administered antifibrinolytic agents. In this case the patient with the thin line would benefit from an antifibrinolytic agent (e.g. tranexamic acid). All common parameters were markedly distinguished between the two patients by our PCFA assay (R-time, Angle, Maximal Amplitude (MA), and % Lysis 30 Minutes after MA (LY30)). Panel B shows standard thromboelastographs (aka “native” thromboelastograhs) of the same normal blood sample (bold line) versus the same hyperfibrinolytic blood sample (thin line) that were not supplemented with plasmin, where no differences are observed after 1 hour (the tracings are virtually identical). Panel C is another PCFA showing normal blood (thin line) versus hyperfibrinolytic blood (bold line), where the hyperfibrinolytic blood does not ever even begin to form a clot. Panel D is the same patient blood samples as in Panel C, where standard thromboelastography shows nearly identical tracings and is unable to diagnose the underlying hyperfibrinolysis in the patient with the bold tracing. All parameters markedly differentiated between healthy and hyperfibrinolytic blood by PCFA compared to standard/native thromboelastographs.

Panel A in FIG. 9 shows the profound power of PCFA used in a thromboelastograph to diagnose hyperfibrinolysis in a patient with severe liver disease immediately prior to undergoing liver transplantation (thin line) relative to a standard “native” thromboelastograph (bold line). Panel B demonstrates that 24 hours after receiving a new liver via transplantation, that same patient's PCFA assay closely mirrors the standard thromboelastograph. Taken together, these results of pre- and post-liver transplantation on PCFA testing versus standard functional coagulation testing with a native thromboelastograph clearly demonstrate the power of PCFA to discriminate hyperfibrinolysis when compared to standard functional coagulation testing. Panel C again demonstrates the power of PCFA to diagnose hyperfibrinolysis in another patient with severe liver disease immediately before undergoing liver transplantation (thin line=PCFA, bold line=standard thromboelastograph), and in Panel D a repeat PCFA 24 hours after undergoing liver transplantation again shows that all the measured parameters corrected to nearly mirror those of a standard thromboelastograph (bold line), again demonstrating the tremendous power of PCFA to discriminate pathological hyperfibrinolysis in comparison to standard functional coagulation testing.

Convention tests lack the capability that certain embodiments of the inventive PCFA does to diagnose pathological hyperfibrinolytic states in such a short time period, which is critical to helping with clinical decision making and personalized care decisions of sick patients at high risk for major bleeding where time is of the essence. Similarly, conventional tests lack the capability of certain embodiments of the inventive PCFA to diagnose reasons for pathological fibrinolysis shutdown states, which is also critical to helping with clinical decision making and personalized care decisions of sick patients. A very conservative estimate of the economic potential would be 50,000 or more PCFA assays performed per year in the United States alone, and likely a very substantial positive economic impact on patients being able to return to the workforce through better clinical outcomes as a result of PCFA testing.

### Example 2

This example describes the effect of certain exogenous plasmin concentration on assay results. A plasmin concentration of 10% of the maximum theoretical amount of plasmin resulted in a shorter reaction time than 0.1% and 1%.

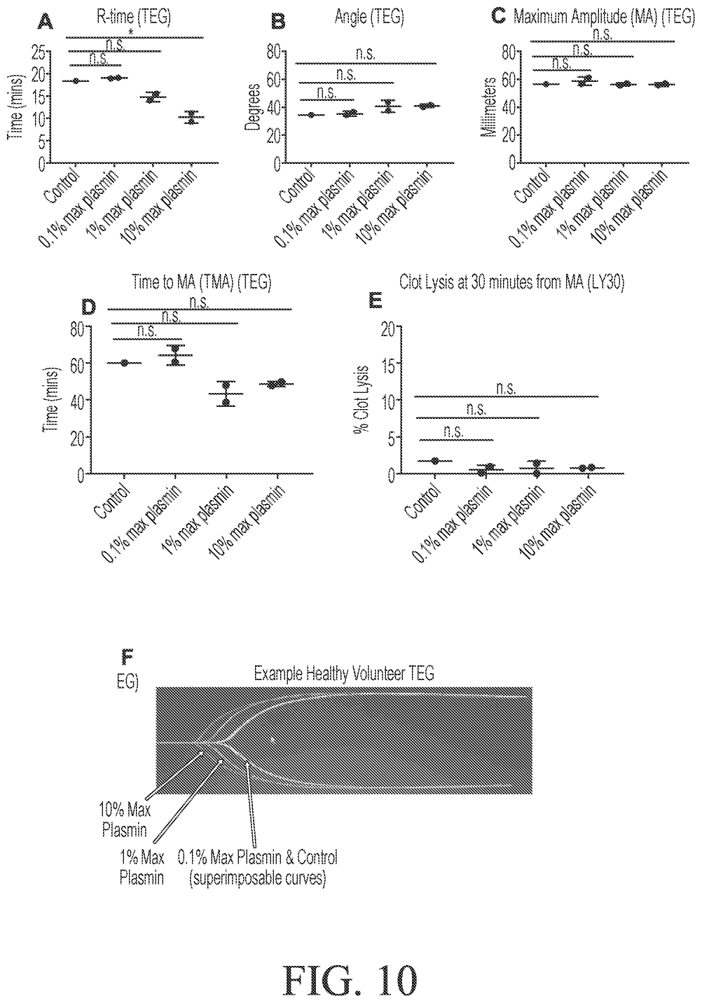

PCFA was run on thromboelastography (TEG) using healthy human whole blood anticoagulated with citrate and mixed with (i) vehicle control, (ii) about 0.1% of the maximum amount of plasmin that can be generated in a human based on average human circulating plasminogen levels (0.166 ug/mL human plasmin), (iii) about 1.0% max plasmin (1.66 ug/mL human plasmin), and (iv) about 10% max plasmin (16.6 ug/mL human plasmin). With the exception of a shorter R-time (the “reaction time”), which was the time it takes for enough blood clot to form to cause a 2 mm deflection of the instrument's detection pin, these levels of plasmin on PCFA did not cause significant changes in the commonly measures TEG parameters as shown in FIG. 10. Results shown as mean+/−SD. *=p<0.05.

### Example 3

This example describes the effect of certain exogenous plasmin concentration on assay results. Plasmin concentrations between 10% and 25% of the maximum theoretical amount of plasmin resulted in fast and sensitive assays.

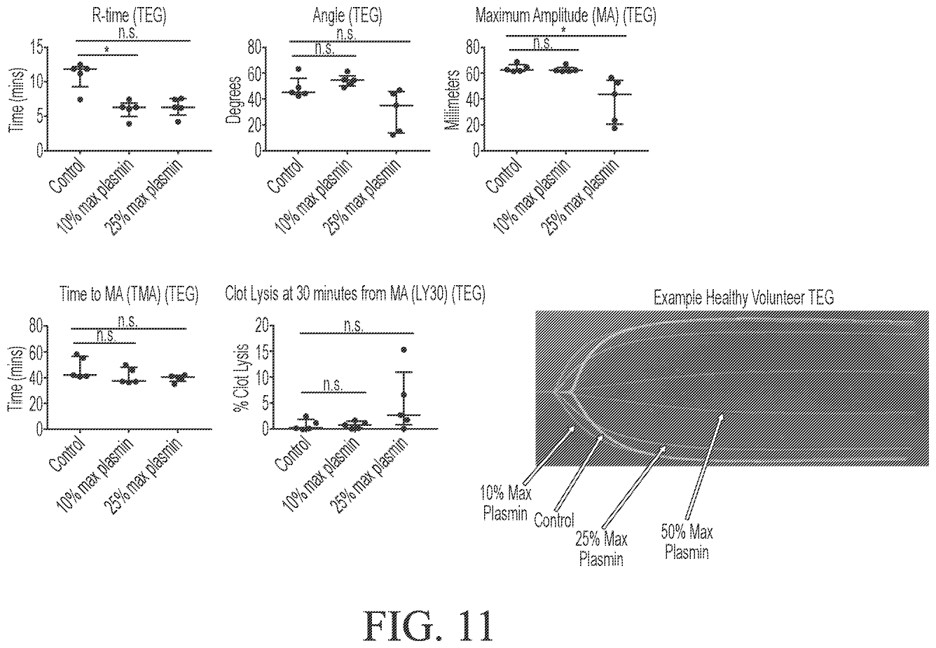

PCFA was run on thromboelastography (TEG) using healthy human whole blood anticoagulated with citrate and mixed with (i) vehicle control, (ii) about 10% of the maximum amount of plasmin that can be generated in a human based on average human circulating plasminogen levels (16.6 ug/mL), (iii) about 25% max plasmin (41.5 ug/mL human plasmin), and (iv) about 50% max plasmin (93 ug/mL human plasmin). The results of the 50% max plasmin are not shown in panels A-E of FIG. 11, because in most cases no blood clot formed and no measurable data was able to be obtained. Beyond the shorter R-time (Panel A), as plasmin concentrations exceeded the about 10% theoretic maximal human plasmin levels there began to be an effect on the strength of the blood clot formed, as evidenced by the reduced maximal amplitude (MA) in the 25% max plasmin group (Panel C) and the 50% max plasmin group, which often did not even form a blood clot in the first place. Results shown as median with interquartile ranges. *=p<0.05.

### Example 4

This example describes the combination of exogenous plasmin and exogenous tPA in a single assay.

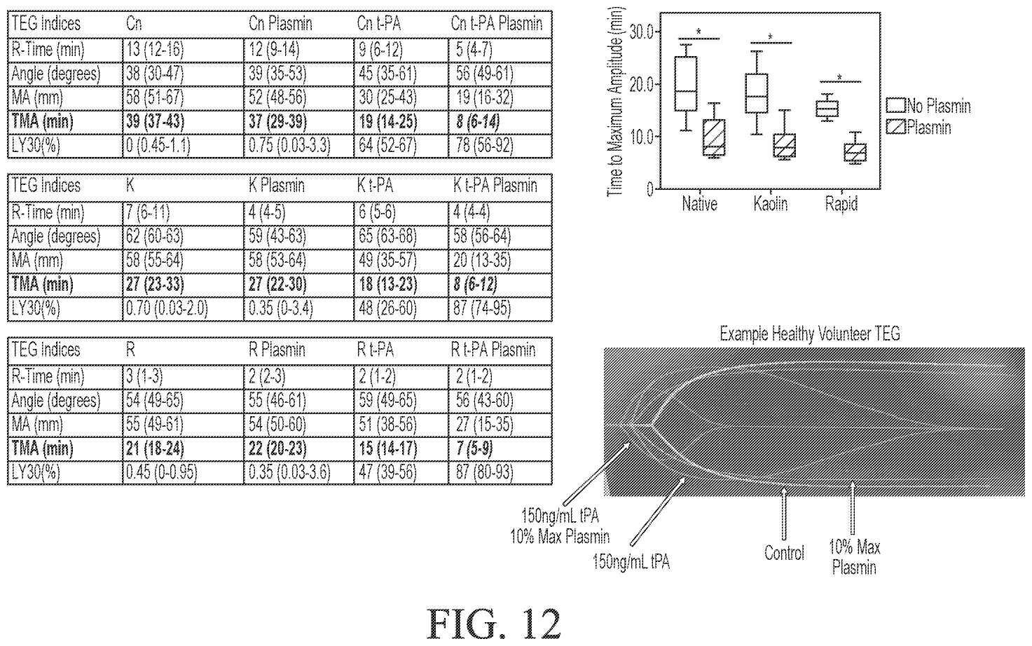

TEG was run using healthy human volunteer whole blood anticoagulated with citrate and mixed with (i) vehicle control, (ii) 150 ng/mL recombinant human tissue-plasminogen activator (t-PA), (iii) about 10% max plasmin (16.6 ug/mL human plasmin), or (iv) a combination of 150 ng/mL t-PA plus about 10% max plasmin. TEGs were performed using citrated native conditions (Cn) as well as with kaolin (K) and RapidTEG™ reagent (R) “activated” conditions. Shortening in time to maximal amplitude (TMA) as well as reduced clot strength (maximal amplitude, or MA) and increased clot lysis 30 minutes after reaching MA (LY30) were observed for all methods of TEG (Cn, K, and R) when t-PA and plasmin were combined in the same assay compared to control, t-PA only, and plasmin only as shown in FIG. 12. t-PA addition to TEG assays has previously been shown to be an extremely sensitive assay for detecting hyperfibrinolysis and predicting massive blood transfusion requirements in trauma patients (H. B. Moore et al. Journal of the American College of Surgeons, 2017 July; 225(1): 138-147), where hyperfibrinolysis, which causes major bleeding, is often manifested after depletion of plasminogen activator inhibitor 1 (PAI-1) due to endogenous plasminogen activator release (e.g. t-PA) amongst other mechanisms (M. P. Chapman et al. J Trauma Acute Care Surg. 2016 January; 80(1):16-23.). Addition of plasmin to functional coagulation and fibrinolysis assays (e.g. TEG) containing t-PA more rapidly unmasked fibrinolysis of blood clots, for example due to PAI-1 depletion as is seen in major traumatic injury, than assays containing t-PA alone, and this may potentially provide information for earlier therapeutic intervention such as treatment with the anti-fibrinolytic drug tranexamic acid. Results shown as median with interquartile ranges. *=p<0.05.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A coagulation and fibrinolysis assay composition comprising a sample being tested and plasmin and/or plasminogen that is exogenous with respect to the sample being tested.

2. A coagulation and fibrinolysis assay kit, comprising:
sample collection container free of a subject sample for testing and configured for containing the subject sample; and
an exogenous plasmin and/or plasminogen.

3. The coagulation and fibrinolysis assay kit of claim 2, wherein the exogenous plasmin and/or plasminogen is contained in the sample container.

4. The coagulation and fibrinolysis assay composition or kit as in any preceding claim, wherein the coagulation and fibrinolysis assay is a viscoelastic assay, optionally a thromboelastography (TEG) based viscoelastic assay.

5. A coagulation and fibrinolysis assay composition or kit as in any preceding claim, wherein the coagulation and fibrinolysis assay is a rotational thromboelastometry based assay.

6. The coagulation and fibrinolysis assay composition or kit as in any preceding claim, wherein the Coagulation and fibrinolysis assay uses a microfluidic device.

7. The coagulation and fibrinolysis assay composition or kit as in any preceding claim, wherein the coagulation and fibrinolysis assay uses a device that measures functional coagulation other than through viscoelastic or microfluidic means.

8. The coagulation and fibrinolysis assay composition or kit as in any preceding claim, wherein the plasmin is a purified enzymatically active plasmin and/or the plasminogen is a purified plasminogen that yields an enzymatically active plasmin.

9. The coagulation and fibrinolysis assay composition or kit of claim 8, wherein the purified plasmin is a recombinant plasmin that has greater than 40% amino acid sequence identity to a naturally occurring plasmin and/or the purified plasminogen is a recombinant plasminogen that has greater than 40% amino acid sequence identity to a naturally occurring plasminogen.

10. The coagulation and fibrinolysis assay composition or kit of claim 8, wherein the purified plasmin is a recombinant plasmin that has greater than 60% amino acid sequence identity to a naturally occurring plasmin and/or the purified plasminogen is a recombinant plasminogen that has greater than 60% amino acid sequence identity to a naturally occurring plasminogen.

11. The coagulation and fibrinolysis assay composition or kit of claim 8, wherein the purified plasmin is a recombinant plasmin that has greater than 80% amino acid sequence identity to a naturally occurring plasmin and/or the purified plasminogen is a recombinant plasminogen that has greater than 80% amino acid sequence identity to a naturally occurring plasminogen.

12. The coagulation and fibrinolysis assay or kit of claim 8, wherein the purified plasmin is a recombinant plasmin that has greater than 90% amino acid sequence identity to a naturally occurring plasmin and/or the purified plasminogen is a recombinant plasminogen that has greater than 90% amino acid sequence identity to a naturally occurring plasminogen.

13. The coagulation and fibrinolysis assay composition or kit as in any of claims 1-8, wherein the plasmin is a mammalian plasmin and/or the plasminogen is a mammalian plasminogen.

14. The coagulation and fibrinolysis assay composition or kit as in claim 13, wherein the purified plasmin is a human plasmin and/or the purified plasminogen is a human plasminogen.

15. A method, comprising:
combining a blood or blood-derived sample from a patient being tested with a plasmin and/or plasminogen that is exogenous with respect to the blood or blood-derived sample; and
performing a coagulation and fibrinolysis assay on the sample.

16. The method according to claim 15, wherein the coagulation and fibrinolysis assay is a viscoelastic assay, optionally a thromboelastography (TEG) based viscoelastic assay.

17. The method according to claim 15, wherein the coagulation and fibrinolysis assay is a rotational thromboelastometry based assay.

18. The method according to claim 15, wherein the coagulation and fibrinolysis assay is a microfluidic device based assay.

19. The method according to claim 15, wherein the coagulation and fibrinolysis assay measures functional coagulation other than through viscoelastic or microfluidic means.

20. The method according to any of claims 15-19 wherein the sample from the patient comprises blood from the patient and wherein the plasmin and/or plasminogen is added to a blood collection vial prior to adding the blood from the patient.

21. The method according to any of claims 15-19 wherein the sample from the patient comprises blood from the patient and wherein the plasmin and/or plasminogen is added to a blood collection vial after adding the blood from the patient.

22. The method according to any of claims 15-19, wherein the assay is performed on a patient sample from a patient with known or suspected liver disease or a patient who has been or is in consideration for being listed for liver transplantation by their medical care team, as well as during liver transplantation and up to 1 week after transplantation.

23. The method according to any of claims 15-19, wherein the assay is performed on a patient sample from a trauma patient within 1 month of the initial trauma.

24. The method according to claim 23, wherein the assay is performed on a patient sample from a trauma patient within 24 hours of the initial trauma.

25. The method according to any of claims 15-23, wherein the assay is analyzed within 90 minutes of initiating the assay and optionally is continuously analyzed for additional information up to 120 minutes thereafter, and provides information regarding the coagulation state of the patient.

26. The method according to claim 25, wherein the assay is analyzed within 15 minutes of initiating the assay and optionally is continuously analyzed for additional information up to 120 minutes thereafter, and provides information regarding the coagulation state of the patient.

27. The method according to any of claims 15-24, wherein the assay measures a narrowing of a graphical output of a viscoelastic assay (thromboelastography or rotational thromboelastometry) where a signal trending towards the horizontal midline is indicative of enhanced fibrinolysis.

28. The method according to any of claims 15-24, wherein the assay measures a narrowing of the graphical output of the viscoelastic assay (thromboelastography or rotational thromboelastometry) where a signal trending towards the horizontal midline to lesser degree than normal is indicative of sub-normal amounts of fibrinolysis (fibrinolysis shutdown), and wherein the assay when performed on a sample from a patient in fibrinolysis shutdown includes exogeneous plasminogen results in a signal trending towards the horizontal midline to a greater degree than for a sample from the same patient but not including the exogeneous plasminogen indicates the fibrinolysis shutdown is due to plasminogen depletion.

29. The method according to any of claims 15-25, wherein the patient is a human patient.

30. A coagulation and fibrinolysis assay composition comprising a sample being tested and an exogenous sequestering agent.

31. A coagulation and fibrinolysis assay kit, comprising:
sample collection container free of a subject sample for testing and configured for containing the subject sample;
an exogenous sequestering agent; and
a clotting activator.

32. A coagulation and fibrinolysis assay composition or kit of claim 31, wherein the exogenous sequestering agent is plasmin, plasminogen, serpin inhibitor, or combinations thereof.

33. The coagulation and fibrinolysis assay composition or kit of any preceding claim, wherein the sample is blood or a blood-derived product.

34. A method, comprising:
combining a blood or blood-derived sample from a patient being tested with a sequestering agent that is exogenous with respect to the blood or blood-derived sample; and
performing a coagulation and fibrinolysis assay on the sample.

35. A method or coagulation and fibrinolysis assay composition or kit of any preceding claim, further comprising a tissue plasminogen activator that is exogenous with respect to the sample.

36. A method, comprising:
combining a blood or blood-derived sample from a patient being tested with a plasmin and/or plasminogen and a tissue plasminogen activator that are exogenous with respect to the blood or blood-derived sample; and
performing a coagulation and fibrinolysis assay on the sample.
